(19) United States
(12) Patent Application Publication
(10) Pub. No.: US 2022/0304595 A1
(43) Pub. Date: Sep. 29, 2022

(54) SHOULDER IMPLANT FOR CENTER OF ROTATION TRACKING

(71) Applicant: Zimmer, Inc., Warsaw, IN (US)

(72) Inventors: Clinton E. Kehres, Warsaw, IN (US); Jeffrey E. Bischoff, Warsaw, IN (US)

(21) Appl. No.: 17/697,603

(22) Filed: Mar. 17, 2022

Related U.S. Application Data
(60) Provisional application No. 63/165,982, filed on Mar. 25, 2021.

(51) Int. Cl. A61B 5/11; A61B 5/00

(52) U.S. Cl. CPC A61B 5/1121; A61B 5/686; A61B 5/4576; A61B 5/4851; A61B 5/1114; A61B 5/742; A61B 2562/0219

(57) ABSTRACT

A sensing system for tracking a center of rotation of a joint can include a computer system including processing circuitry configured to perform operations including: retrieve a first data set collected by a sensor device configured to be implanted into a patient in a fixed location on or within a first bone of the joint, the sensor device configured to collect data associated with movement of the first bone of the joint at a first time, retrieve a second data set collected by the sensor device at a second time subsequent to the first time; analyze the first and the second data sets to calculate first and second center of rotation locations; and compare the first and second center of rotation locations to track migration in the center of rotation of the joint over time.

### CLAIM OF PRIORITY

This application claims the benefit of U.S. Provisional Patent Application Ser. No. 63/165,982, filed on Mar. 25, 2021, the benefit of priority of which is claimed hereby, and which is incorporated by reference herein in its entirety.

### BACKGROUND

The shoulder (glenohumeral) joint is the most mobile joint in the human body. The scapula, clavicle and the humerus all converge to enable a complex range of movements. In a properly functioning shoulder joint, the head of the humerus fits into a shallow socket in the scapula, often referred to as the glenoid or the glenoid fossa. The head of the humerus articulates at least partially within the glenoid during movement of the shoulder joint. The structure of the mating surfaces of the humeral head and the glenoid, together with various surrounding connective or supporting tissues, allow the shoulder joint to freely articulate through a wide range of motion, at least in a healthy shoulder joint.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a sensing system including a sensor device, in accordance with at least one example of the present application.

FIG. 2 illustrates a prosthetic shoulder joint including a sensor device, in accordance with at least one example of the present application.

FIG. 3A illustrates a block diagram of a sensor device, in accordance with at least one example of the present application.

FIG. 3B illustrates a block diagram of a transmitter device, in accordance with at least one example of the present application.

FIG. 4A illustrates a flowchart showing a method of estimating a position and orientation of a sensor device, in accordance with at least one example of the present application.

FIG. 4B illustrates a data set including various three-dimensional data points, in accordance with at least one example of the present application.

FIG. 5A illustrates a best fit model used for estimating a first center of rotation location of a shoulder joint, in accordance with at least one example of the present application.

FIG. 5B illustrates a graphical representation of a first center of rotation location of a shoulder joint, in accordance with at least one example of the present application.

FIG. 6 illustrates a flowchart showing a method for tracking a center of rotation of a joint, in accordance with at least one example of the present application.

FIG. 7 illustrates an example architecture and componentry for a sensing system, in accordance with at least one example of the present application.

FIG. 8 illustrates a block diagram of an example machine upon which any one or more of the techniques discussed herein can be performed, in accordance with at least one example of the present application.

### DETAILED DESCRIPTION

The shoulder joint includes numerous types of soft tissues, such as connective or supporting tissues including articular cartilage, ligaments, joint capsules, and bursa. These soft tissues can undergo various degenerative changes over time, such as caused by rheumatoid arthritis, osteoarthritis, vascular necrosis, bone fracture, or trauma resulting from an injury. When severe damage occurs and no other means of treatment are found to be effective, a total or partial shoulder replacement (shoulder arthroplasty) can become necessary to alleviate a patient's pain and to restore some or all of the natural range of movement of the shoulder joint.

Total shoulder replacements involve the implantation of an artificial glenohumeral joint, such as including the implantation of a prosthetic humeral component and a prosthetic glenoid component. The humeral component replaces the natural humeral head and includes a stem portion and a head portion. The glenoid component includes an articulating cup shaped to receive the head portion of the humeral component. The glenoid is typically first resurfaced to prepare the glenoid to receive the glenoid component. The prosthetic humeral and glenoid components of the artificial joint are matched with the bio-kinematics of the patient, in an effort to maintain or restore the normal and wide range of motion of a healthy shoulder joint. While total shoulder replacement can correct numerous shoulder joint issues, it cannot alleviate degenerative conditions of the rotator cuff, such as rotator cuff tear arthropathy.

A reverse shoulder replacement (reverse shoulder arthroplasty) can be performed to correct rotator cuff arthropathy. A reverse shoulder replacement involves a different set of prosthetic humeral and glenoid components relative to those used in a total shoulder replacement. In a reverse shoulder replacement, the humeral component includes an articulating cup attached to a stem that is implanted into the humerus, and the glenoid implant includes a spherical component used to provide an articular surface for the humeral cup. A physician can use various imaging techniques such as X-ray, CT, or MRI to visualize rotator cuff damage. However, the rotator cuff can slowly degenerate over a lengthy period of time, such as over years or decades.

During this period, it can be difficult to accurately determine the health of the shoulder joint, as relatively minor changes can be difficult to identify. Further, repeated clinical visits may not be practical for a patient, either logistically or economically. As a result, a patient can often suffer pain and reduced mobility of the shoulder joint for much longer than necessary before seeking a corrective procedure such as a reverse shoulder replacement. As the rotator cuff and related soft tissues progressively weaken, the head of the humerus, or a head portion of a prosthetic humeral component, migrates away from the glenoid, or a prosthetic glenoid component. As such, progressive degeneration of the rotator cuff and related soft tissues can be accurately monitored by tracking migration of the center of rotation location of a natural or prosthetic shoulder joint.

The invention discussed herein can help to address the above issues, among others, such as by providing a sensing system capable of allowing healthcare personnel, and individual patients, to accurately monitor the condition of the patient's shoulder joint periodically and/or remotely. For example, the sensing system can include an implantable sensor device capable of generating positional data, such as corresponding to a range of motion of the shoulder joint. The data can then be analyzed to determine the center of rotation location of the shoulder joint. The present inventors have recognized that the availability of compact and implantable sensing technology and miniaturized electronic circuitry, such as for generating acceleration and rate of rotation data, and for wireless communication, can enable a new sensing system capable of generating new and clinically relevant data from a location within a patient.

For example, the sensor device can include an inertial measurement unit (IMU) and can be implanted directly on or within the humerus. The sensor device can be activated periodically to collect data, such as at fixed intervals over a number of months or years. The data can then be analyzed to periodically calculate the center of rotation location of the humerus relative to the location of the glenoid. Over time, the various center of rotation locations can be compared to accurately the track migration of the humeral head away from the glenoid. Accordingly, the sensor device can allow a physician to easily, and precisely, monitor the condition of a patient's rotator cuff and related soft tissues, without the need for repeated clinical imaging visits. As a result, the physician can provide a recommendation for a corrective procedure to a patient at a time likely much earlier than a patient would otherwise seek help. Moreover, individual patients' data can be aggregated to develop a reference database, such as to aid physicians in predictive assessment of various progressive and degenerative shoulder joint conditions.

While the above overview discusses issues and procedures specific to shoulder replacement procedures, discussion of the following systems, devices, or methods are also applicable for use in the assessment and monitoring of other joints, such in tracking the center of rotation location of the hip (acetabulofemoral) joint. The above overview is intended to provide an overview of subject matter of the present patent application. It is not intended to provide an exclusive or exhaustive explanation of the invention. The description below is included to provide further information about the present patent application.

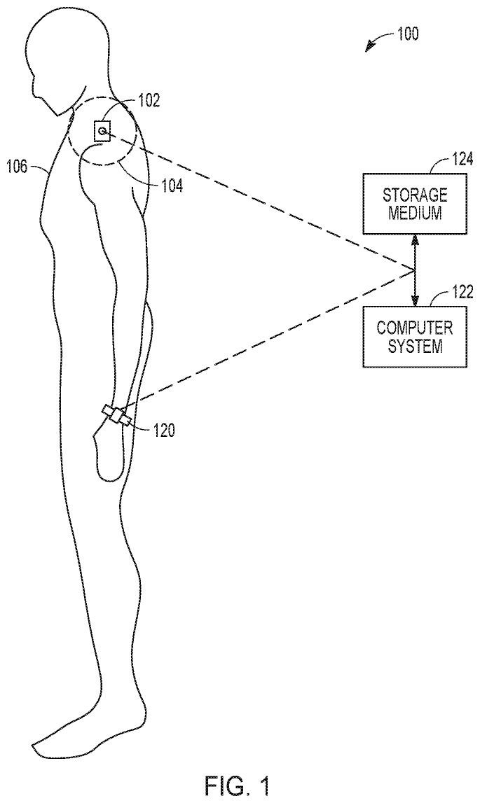

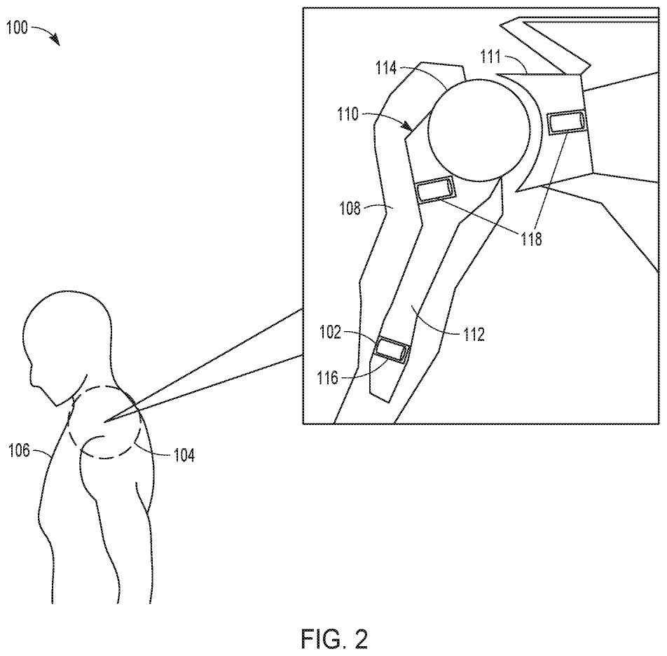

FIG. 1 illustrates a sensing system 100 including a sensor device 102, in accordance with at least one example of the present application. FIG. 2 illustrates a prosthetic shoulder joint 104 including a sensor device 102, in accordance with at least one example of the present application. FIGS. 1-2 are discussed below concurrently. The sensor device 102 can include one or more sensors to generate positional or spatial location data. The sensor device 102 can be surgically implanted at various fixed locations on or within a first bone associated with the shoulder joint 104 of a patient 106. For example, the sensor device 102 can be positioned on or within a humerus 108, such by being rigidly affixed to bone, or positioned on or within a humeral component 110 implanted into the patient 106. In an example, the sensor device 102 can be implanted and anchored into bone in a position generally below or underneath a prosthetic humeral implant, such as humeral component 110.

The humeral component 110 can extend at least partially within and along a length of the humerus 108. The humeral component 110 can include a stem portion 112 and a head portion 114. The sensor device 102 can be fixedly coupled to the stem portion 112, such as via snap fit, an adhesive, or various types of welding including, but not limited, vibration welding. The sensor device 102 can be fixedly located within the stem portion 112 or within the stem portion 112, such by positioning and securing the sensor device 102 within a cavity 116 defined by the stem portion 112. The approximate location of the sensor device 102, relative to the humerus 108 or the humeral component 110, can be selected by a physician based on various properties of a patient's bone, the type and size of the sensor device 102, or the type and size of an implant, such as the humeral component 110.

The sensing system 100 can include a second sensor device 118. In such an example, the sensor device 102 can be a first sensor device. The second sensor device 118 can be similar to the sensor device 102; but can be implanted at various different fixed locations, relative to the sensor device 102, on or within the humerus 108, humeral component 110, or other bones or bone surfaces included in or near the shoulder joint 104. For example, the second sensor device 118 can be implanted on or within a scapula 111, glenoid, or a prosthetic glenoid component, such as a glenosphere, of the patient 106. The size and shape of the second sensor device 118 can be altered relative to the sensor device 102, such as to help to dimensionally conform to, or fit within, the scapula 111. The second sensor device 118 can also be located at a generally opposite portion of the humerus 108, relative to the sensor device 102. In some examples, the sensing system 100 can include three sensor devices, such as a first and a second sensor device on or within the humerus, and a third sensor device on or within the scapula 111.

The sensing system 100 can include a transmitter device 120. The transmitter device 120 can be operably coupled to the sensor device 102. For example, the transmitter device 120 can wirelessly power, read, and control the sensor device 102. The transmitter device 120 can transmit data generated by the sensor device 102, such as to a separate computer system or cloud service for storage. In some examples, the transmitter device 120 can aggregate data, such as by combining data generated by the sensor device 102 and the second sensor device 118. In some examples, the transmitter device 120 can be a consumer electronic device, such as a Fitbit®, a Jawbone®, an Apple Watch®, or a mobile phone located externally to the patient 106. In other examples, the transmitter device 120 can be a custom device located externally to the patient 106. The transmitter device 120 can be worn on or about the patient 106, such as around a wrist or upper arm of the patient 106. The transmitter device 120 can otherwise be temporarily attached to the patient 106 in any number of external locations, such as via temporary adhesive. The sensing system 100 can thereby generate and collect, aggregate, and transmit data corresponding to movement of the humerus 108 from a location internal to the patient 106.

The sensing system 100 can include computer system 122. The computer system 122 can be, for example, a mobile phone or other mobile device. In such an example, the transmitter device 120 can be mobile phone and can include the computer system 122. The computer system 122 can be a consumer computer system such as a personal laptop or a desktop computer, or a professional computer system, such as located at a clinic, hospital, or other point of healthcare. The sensing system 100 can include a data repository 124. In some examples, the data repository 124 can be a physical memory of the computer system 122, a cloud service, or other types of remote computer-readable storage mediums, such as included in a separate server. In various examples, any of the sensor device 102, the reference sensor the transmitter device 120, or the computer system 122 can be in network communication with the data repository 124, such as to transmit data generated by the sensor device 102 to the data repository 124.

The computer system 122 can analyze data generated by the sensor device 102, such as by utilizing various algorithms or functions implemented in a mobile application or other software programs, to calculate a center of rotation location of the shoulder joint 104. Over time, the computer system 122 can periodically calculate the center of rotation from data collected by the sensor device 102 at a subsequent time, such as in specified fixed intervals over a period of years. The computer system 122 can then compare the original center of rotation location to subsequent center of rotation locations. For example, the computer system 122 can plot various center of rotation locations on a graphical representation of a glenoid, such as to illustrate migration of the humerus 108 away from the glenoid.

In the operation of some examples, the sensor device 102 can be implanted into the humerus 108 of a patient 106. The sensor device 102 can be activated periodically, such as via a user interface of the computer system 122, to collect data sets corresponding to or associated with movement of the humerus 108, during a specified timeframe. The transmitter device 120 can obtain the data sets from the sensor device 102 to calculate the center of rotation location of each data set. The computer system 122 can then map the center rotation locations relative to one another to monitor progressive migration in the center of rotation of the shoulder joint 104. The center of rotation locations can be stored on the data repository 124 and can be retrieved remotely, such as by a physician.

The sensing system 100 can provide a number of benefits to both a patient and to physician. The sensing system 100 can allow a physician to periodically, and accurately, assess the extent of rotator cuff degradation and related tissues from a location remote from a patient. This can help to reduce expenditure for a patient associated with repeated clinic imaging, and partially, or entirely, eliminate the inconveniences associated with repeated clinical visits. Further, the sensing system 100 can help to improve functional outcomes for a patient by providing an early indication into the condition of a natural or replacement shoulder joint, such as to prevent significant bone erosion or malalignment, after which point corrective surgical options can become more limited.

As a result, this can help to significantly reduce the amount of pain and debilitation suffered by a patient both before and after the patient seeks the aid of a physician for corrective action. Moreover, the data collected from multiple patients can be used to establish a reference database, such as to allow an individual patient's data to be benchmarked against data collected from other patients. This can help to improve a physician's ability to predict and understand progressive joint deterioration, such as to aid a physician in recommending treatment options or corrective procedure to future patients.

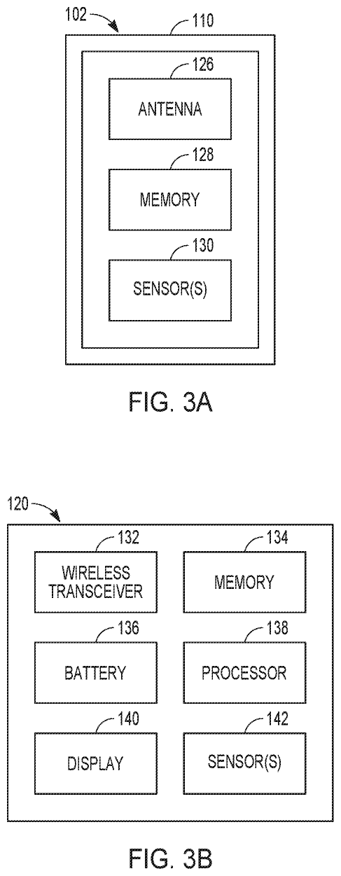

FIG. 3A illustrates a block diagram of a sensor device 102, in accordance with at least one example of the present application. FIG. 3B illustrates a block diagram of a transmitter device 120, in accordance with at least one example of the present application. FIGS. 3A-3B are discussed below concurrently. As shown in FIG. 3A, the sensor device 102 can be a passive device, such as, but not limited to, a passive RFID tag or transponder. For example, the sensor device 102 can be a metal-mount RFID tag, such as to help mitigate issues around metallic objects, such as when located on or within the humeral component 110.

As shown in FIG. 3A, the sensor device 102 can include any of an antenna 126, a memory 128, and a sensor 130. The antenna 126 can be, for example, a combination RFID receiver and a radio frequency power harvesting system, such as rectenna, that allows RF power or alternating current (AC) to be converted into usable DC energy by the sensor device 102. The antenna 126 can allow the sensor device 102 to be wirelessly powered, interrogated, or otherwise controlled in the presence of an external device, such as the transmitter device 120 or computer system 122. The memory 128 can be a physical storage medium, such as an internal microchip or an integrated circuit (IC). The memory 128 can temporarily store data generated by the sensor 130, such as in a data buffer.

The sensor 130 can be an IMU including an accelerometer, or an IMU including both an accelerometer and a gyroscope, such as to output both three-axis acceleration and rate of rotation (e.g., angular velocity) data, respectively. The sensor 130 can include multiple IMUs or other position sensing technologies, such as magnetic or ultrasonic sensors. For example, the sensor 130 can include, a three-axis compass or magnetometer, or an ultrasonic receiver for use with an external ultrasonic interrogation system, such as included in the transmitter device 120. The transmitter device 120 can be, or otherwise include, various internal components or modules of existing consumer electronic device, such as a Fitbit, a Jawbone, an Apple Watch, or a mobile phone. The transmitter device 120 can be a custom device located externally to the sensor device 102, and to the patient 106.

As shown in FIG. 3B, the transmitter device 120 can include, but not limited to, any of a wireless transceiver 132, a memory 134, a battery 136, a processor 138, a display 140, sensor(s) 142, or still other features relevant to related or unrelated functions of the transmitter device 120. The wireless transceiver 132 can interrogate the sensor device 102 via the antenna 126, such as to read the memory 128 of the sensor device 102 using various wireless protocols, such as near field communication (NFC). In an example, the wireless transceiver 132 can be configured to read the memory 128 of the sensor device 102 from a range of about 1-10 cm, 0.1-1 m, or 1-30 m. The reading range of the wireless transceiver 132 can be dependent, for example, on the frequency or wavelength of the RFID tagging of the sensor device 102, such as VHF, UHF, HF, or LF ranges.

In some examples, the sensing system 100 can include the sensor device 102 and the second sensor device 118. The transmitter device 120 can receive and aggregate the data on the memory 134 of the transmitter device 120, such as to prepare a data set to transmit to the computer system 122 or to the data repository 124. The memory 134 can be a permanent memory, such as RAM or an HHD or SSD, or a removable storage medium, such as a memory card. Data collected from the sensor device 102 can be stored in a fixed location, such as in hardware of the memory 134, or in a virtual data buffer in software, such as pointing at a location on the memory 134. The transmitter device 120 can then transmit data collected on the memory 134 via Bluetooth (e.g., Bluetooth Low Energy), 3GPP LTE, WiFi, near field communication (NFC), or another healthcare compliant communication protocol, to a remote location, such as the data repository 124 for permanent storage. The battery 136 can be a replaceable and rechargeable battery. The processor 138 can receive and execute computer-readable instructions from the computer system 122.

For example, the processor 138 can receive and implement instructions such as to cause the transmitter device 120 to periodically retrieve a data set from the sensor device 102. In some examples, the transmitter device 120 can include the computer system 122. In such an example, the processor 138 can execute all, or part, of the instructions used to calculate a center of rotation location of the shoulder joint 104 from data collected by the sensor device 102. In some examples, the computer system 122 can be a mobile phone, and data can be analyzed in a mobile application on the computer system 122. In other examples, the computer system 122 can be a computer, such as a laptop or desktop computer, a remote server, or other devices, and data can analyzed in a software program.

In some examples, the transmitter device 120 can automatically remove data from the sensor device 102, such when the memory 128 is filled to capacity. The transmitter device 120 can deliver an alert or other indication to a user upon receiving a confirmation that data from the memory 128 was successfully transferred to the memory 134, or to the data repository 124. Upon receiving the confirmation, the transmitter device 120 can automatically erase the memory 128 of the sensor device 102. The transmitter device 120 can also remove personally identifiable information from data before transmission to remote storage, such as upon receiving an indication that the data repository 124 does not have permission to access personally identifiable information of a patient.

The sensor device 102 can collect data when in an active state. For example, the sensor device 102 can be in an active state when the transmitter device 120 is powering the sensor device. Activation of the sensor device 102 can be based on a user input to the transmitter device 120, or in some examples, the computer system 122, such as via a display device (e.g., user interface) or other physical input features. In some examples, the user input can include entering an activation code. The user input can also be placing the transmitter device 120 within communication range of the sensor device 102. In such an example, activation of the sensor device 102 can be an automatic response to detecting an indication that the sensor device 102 is within a communication range of the transmitter device 120. The sensing system 100 can be configured by a user to collect data during a periodically reoccurring and specified timeframe, such as to periodically collect a data set over fixed time intervals. For example, continuous data collection can occur during about daily, bi-monthly, monthly, or yearly fixed intervals. In various examples, the meaning of the term “collect” can include any all of generating, storing, aggregating, or transmitting data, such as executable by various componentry of the sensing system 100 including the sensor device 102, the transmitter device 120, or the computer system 122.

The transmitter device 120 can also be configured transmit data received from the sensor device to a remote storage location, such as the data repository 124, during a periodically reoccurring and specified timeframe. For example, the transmitter device 120 can transmit data to storage in about 5-10-minute intervals, 11-59-minute intervals, hourly intervals, or daily intervals. In some examples, any of the sensor device 102 or second sensor device 118 can be an active device, such as including any of the components and able to perform any of the functions of, the transmitter device 120 or the computer system 122. The sensing system 100 can include any number of different portable electronic mobile devices, including cellular phones, personal digital assistants (PDA's), laptop computers, portable gaming devices, portable media players, e-book readers, watches, as well as non-portable devices such as desktop computers.

The sensor device 102 and the transmitter device 120 depicted in FIGS. 3A-3B are merely illustrative, and other sensor or transmitting devices can be employed, and in other locations, in accordance with this disclosure. For example, other technology including in, or usable with, the sensing system 100 including the sensor device 102, the transmitter device 120, or the computer system 122, can include ultrasonic/ultrasound devices (e.g., with an internal receiver and an external interrogation device), magnetic markers (e.g., spatial magnetic interrogation), other markers or sensor that can be externally interrogated such with global navigation satellite system (GNSS), or an ultrawideband (UWB) system, or automatic identification technology to recognize specific movements of the shoulder joint 104. In some examples, data collected by the sensor device 102 can be input into a data analytics or other computer-implemented systems for developing predictive analytics.

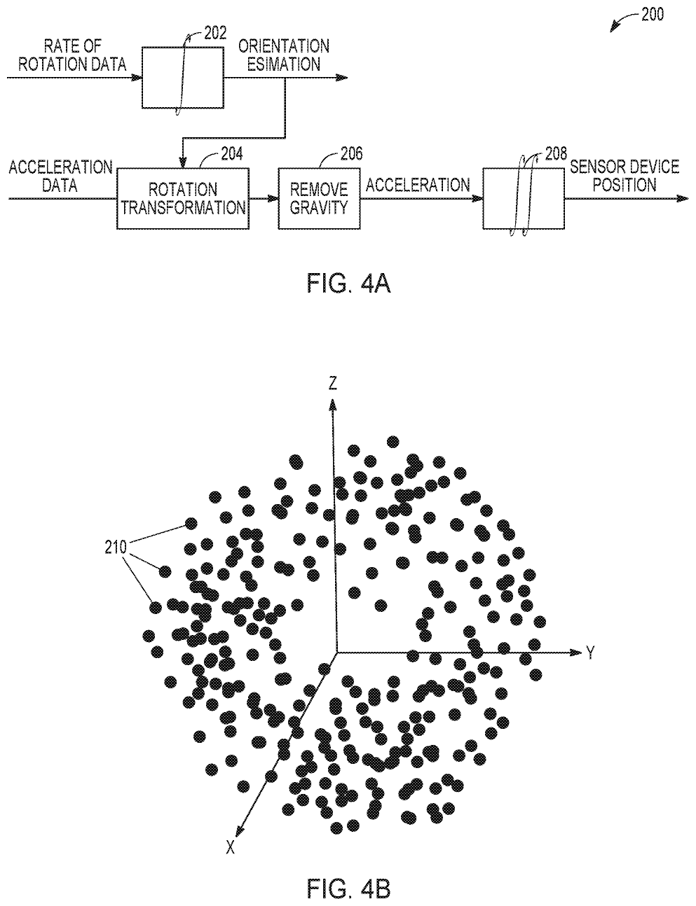

FIG. 4A illustrates a flowchart showing a method 200 of estimating a position and orientation of a sensor device, in accordance with at least one example of the present application. FIG. 4B illustrates a data set including various three-dimensional data points. FIGS. 4A-4B are discussed below concurrently; and are discussed with reference to the sensing system 100 shown and described in FIGS. 1-3B above. The sensing system 100 can generally be an inertial navigation system (INS), such as including the sensor device 102 to collect accelerometer and a gyroscope data via an IMU, and the computer system 122 to continuously or periodically calculate an estimate position and orientation of the humerus 108 from accelerometer and a gyroscope data.

In the field of inertial navigation, a number of methods are known and used to estimate a position and orientation of a movable object containing an IMU or INS. Accordingly, the computer system 122 can implement any of a variety of approaches, implementing different algorithms or functions, to track motion of the sensor device 102. For example, as shown in FIG. 4A, the sensing system 100 can use dead reckoning (e.g., inertial integration without corrective input from external devices) to track motion of the sensor device 102, and correspondingly, the humerus 108, such as from a data set collected by the sensor device 102 during a specified timeframe.

The method 200 is a basic example of how a dead reckoning approach can be implemented by the computer system 122, to track the sensor device 102 in three-dimensional space. The method 200 can begin with operation 202. Operation 202 includes the integration of rate of rotation data (e.g., angular velocity) collected by a gyroscope of the sensor device 102. The integration of gyroscopic data can provide an orientation estimate for the sensor device 102 at a given point in time. Once the orientation of the sensor device 102 is known, acceleration data collected by the accelerometer of sensor device 102 can be transformed at operation 204.

Operation 204 can be the rotation transformation needed to relate two inertial frames. For example, a first inertial frame can be the inertial frame in which the sensor device 102 operates in, and a second inertial frame can be a fixed, external reference frame not subject to accelerative or rotational force data that the sensor device 102 is configured to collect. For example, the second inertial frame can be a fixed location point on or within the patient, such on the scapula, from which the linear distance between the sensor device 102 and the fixed locational point is measured at the time the sensor device 102 is implanted. The second inertial frame can thereby be used to calibrate the sensor device 102, such as to relate the inertial frame of the sensor device to the orientation of the center of the glenoid. The second inertial frame can also include a z-axis extending orthogonally to the Earth's surface to relate data to various directional labels such as “down” and “up”.

In some examples, any acceleration due to the Earth's gravity can be subtracted or otherwise removed at optional operation 206, such as by using data generated by a magnetometer of the sensor device 102 to measure the direction and force a magnetic field. A magnetometer can also help to filter out positional errors due to noise and help to compensate for integration drift (e.g., positional drift). Finally, at operation 208, double integration of the transformed acceleration data can provide an estimate position for the sensor device 102 in three-dimensional space at a given point in time. In some examples, such as during any of operations 202-208, the sensor device 102 can be brought into a known positional relationship relative to the transmitter device 120 to avoid or to help correct positional drift. For example, the transmitter device 120 can include GPS functionality, and can deliver an alert or instruction to move the transmitter device 120 into the known positional relationship, such as an arm's length away from the sensor device 102.

The method 200 can thereby be used to generate a plurality of data points 210, such as from a plurality of selected points in time collected by the sensor device 102 during a specified timeframe (e.g., from a single data set). The data points 210 can be three-dimensional positional or locational coordinates. In some examples, each of the data points 210 calculated can be weighted or non-weighted running averages, such as manually or automatically identified or selected from data, to improve the accuracy of each data point 210 generated. In some examples, the sensing system 100 can include two or more sensors located in a fixed position relative to the humerus 108, such as the sensor device 102 and the second sensor device 118. In such an example, each data point 210 can be calculated using data aggregated from both the sensor device 102 and the second sensor device 118. Moreover, further processing of data such as optimization-smoothing and filtering, Kalman filtering, or complementary filtering, can be implemented by the computer system 122.

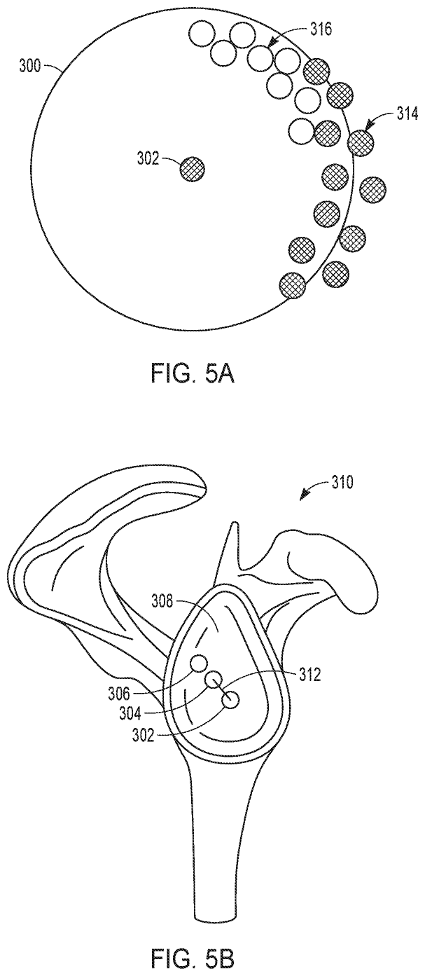

In some examples, the sensor device may not include a gyroscope. In such an example, the plurality of data points 210 (e.g., location coordinates) can be generated from acceleration data alone (e.g., translation from or relative to a known geospatial location), without the use of orientation information. The known geospatial location can be a fixed location on or within a patient's glenoid, such as a location of the second sensor device 118 on or within the scapula 111. A known distance from the location or position of the sensor device 102 to the second sensor device 118, such as obtained during implantation of the sensor device 102 and the second sensor device 118, can thereby to allow the computer system 122 to interpret movement of the humerus 108 relative to the scapula 111. In such an example, the method 200 can instead begin at operation 206 or operation 208. The orientation of the sensor device 102, and accordingly, the humerus 108 or the humeral component 110, can then be determined using an estimated center of rotation location (discussed with regard to FIGS. 5A-5B below). For example, a line or vector can be drawn between an individual data point and the estimated center of rotation location to deduce or assume the orientation of the sensor device 102 using pure rotation relative to the second sensor device 118 fixedly located on or within the scapula 111. However, while the orientation of the sensor device 102 is not required to track the location or position of the sensor device 102 to estimate a center of rotation location of the shoulder joint 104, tracking the internal and external rotation of the sensor device 102 can be helpful in preparing for a reverse shoulder replacement or total shoulder replacement procedures, as pure rotation cannot be assumed when measuring for such an operation. As such, tracking the orientation of a head of the humerus 108, or the head portion 114 of the humeral component 110, relative to the glenoid fossa of the scapula 111, can eliminate the need for further data collection, such obtained during one or more clinical visits.

Any number of data points 210 can be generated from a data set and can be plotted on a three-axis graph, as shown in FIG. 4B. As previously discussed above, the sensing system 100 can collect data sets during a specified timeframe, such as over days, months, or years. As such, the data points 210 can correspond to and illustrate a range of motion that the humerus 108 experiences during everyday activities of the patient 106. The data set 200 can also include data points 210 generated at various locations during a specified motion pattern, such moving an arm of the patient 106 through a maximum range of motion of the shoulder joint 104 through. The maximum range of motion can include, for example, a maximum adduction/abduction, flexion/extension, or internal/external rotation of the shoulder joint 104.

The transmitter device 120 or the computer system 122 can include a display device (e.g., user interface) to show an animation, or an image, of a specific motion pattern to a user during active data collection via the sensor device 102. For example, a user can select an image of a patient, and a specific path of motion, such as for an arm, can be shown on the display device in response. Additionally, sensing system 100 can track various excursions or movements of a joint to identify, for example, a region of weakness or instability of the joint subject to increased risk of dislocation or subluxation, such as during high abduction or adduction. The sensing system 100 such as via the transmitter device 120 or the computer system 122, can then provide an audible real-time alert or other cautionary feedback to a patient during movements causing the shoulder joint 104 to approach, encroach on, or enter the identified region or regions of instability.

FIG. 5A illustrates a best fit model 300 used for estimating a first center of rotation location of a shoulder joint, in accordance with at least one example of the present application. FIG. 5B illustrates a graphical representation 310 of a first center of rotation location 302 of a shoulder joint, in accordance with at least one example of the present application. FIGS. 5A-5B are discussed below concurrently. As discussed in FIGS. 4A-4B above, the computer system 122 can analyze a data set collected by the sensor device 102 to generate a plurality of data points 210, such as corresponding to coordinates in three-dimensional space.

The computer system 122 can further analyze a data set to find a best fit model 300 for a data set, and subsequently, calculate the first center of rotation location 302 for the shoulder joint 104 based on the best fit model 300. The computer system 122 can implement any of a variety of approaches, including different algorithms or functions, to generate the best fit model 300. In one example, a two-step combination of singular value decomposition (SVD) and the method of least-squares can be implemented to calculate the center of rotation location 302. First, singular value decomposition (SVD) can be used to find a two-dimensional plane that best fits a set of data points in three-dimensional space, such as the data points 210 of a data set shown in FIG. 4B. The data points can then be projected onto the two-dimensional plane to obtain new, two-dimensional planar coordinates for each data point.

Second, the method of least-squares can be used to fit a two-dimension circle (e.g., best fit model 300) to the two-dimensional planar coordinates. The two-dimensional arc or circle can then be projected back onto the original three-dimensional graph to obtain three-dimensional positional coordinates for the best fit model 300. The computer system 122 can then find the location of the center of the best fit model 300 to calculate the first center of rotation location 302. Alternatively, spherical regression can be used to generate a three-dimensional spherical best fit model rather than a two-dimensional circular best fit model 300. The meaning of “best fit” can thereby mean be circle or sphere that minimizes the sum of squared distances from a plurality of data points to an outer surface or the circle or sphere. Other methods or approaches, such as fitting an ellipsoid model to data points can also be used.

In some examples, the sensing system 100 can include two or more sensors, such as the sensor device 102 and the second sensor device 118. The sensor device 102 can be located on or within the humerus 108 or the humeral component 110 and the second sensor device 118 can be located on or within the scapula 111. Such an arrangement can help to allow improve the tracking and evaluation of the humerus 108 or humeral component 110 relative to the scapula 111. In some examples, the sensor device 102 and the second sensor device 118 can be located in different fixed positions relative to each other on or within the humerus 108 or the humeral component 110, or the sensing system 100 can further include a third sensor device to allow for two sensor devices on or within the humerus 108 or the humeral component 110, and an additional sensor device on or within the scapula 111.

In such examples, a center of rotation of the humerus 108 humeral component 110 can be calculated according in accordance with US Patent Publication No.: 2018/0085171A1, titled COMPUTER-ASSISTED SURGERY SYSTEM AND METHOD FOR CALCULATING A DISTANCE WITH INERTIAL SENSORS, herein incorporated by reference in its entirety. In some examples, methods described in U.S. Pat. No. 7,427,272 titled: METHOD FOR LOCATING THE MECHANICAL AXIS OF A FEMUR, herein incorporated by the reference in its entirety can also be implemented.

The approaches discussed above are simply several of many potential mechanisms for implementing inertial navigation based on implantable sensors, such as usable to track a center of rotation of a bone or joint from acceleration and rate of rotation data generated by an IMU. Other techniques or algorithms can be used to calculate the center of rotation of a bone or joint from data generated by three-axis, six, or nine-axis IMUs, in accordance with this disclosure.

With regard to FIG. 5B, any number of center of rotation locations can be calculated from any number of data sets collected by the sensor device 102. Multiple center of rotation locations can then be compared by the computer system 122 to track migration in a center of rotation of the humerus 108, and accordingly, degeneration of the shoulder joint 104 joint overtime. For example, the first center of rotation location 302 can be a center of rotation location calculated from a first data set. A second center of rotation location 304 and a third center of rotation location 306 can also be calculated from a second and a third data set, respectively (hereinafter the “first location”, “second location” and “third location”). The first 302, second 304, and third 306 center of rotation locations can be mapped relative to one another and relative a glenoid 308, such as to create a graphical representation 310 of migration in the center of rotation of the humerus 108 over time.

In an example, the first center of rotation location 302 can represent a calculated center of rotation of the humerus 108 relative to the glenoid 308 at a first time. The first time can be a specified timeframe beginning immediately after an operation implanting the sensor device 102, such as a total shoulder replacement procedure. For example, a physician can activate the sensing system 100 to calculate a first center of rotation location from a first data set collected during the first time, to record a reference center of rotation location where the humerus 108 is centered on the glenoid 308. The second center of rotation location 304 can be calculated at, for example, a second time about 3-5, 6-8, or 9-15 years after the first center of rotation location. The third center of rotation location 306 can be, for example, calculated at a third time about 16-20, 21-25, or 26-30 years after the second center of rotation location 304. Various other center of rotation locations can be calculated between or after the first 302, second 304, or third 306 center of rotation locations. The other center of rotation locations can all be directly mapped, or can be first be selectively filtered, such as to map only incremental or significant shifts in the center of rotation of the shoulder joint 104 over time. The specified timeframes discussed above are merely exemplary, and shorter or longer timeframes can also be utilized in accordance with the disclosure.

Mapping the first 302, second 304, and third 306 center of rotation locations can include many other mechanisms for displaying or quantifying data. For example, the first 302, second 304, and third 306 center of rotation locations can be color coded, as such being displayed in different colors relative to one another. For example, the graphical representation 310 be a moving graphical representation or animation displayable to a user on a display device (e.g., user interface) of the computer system 122, such as showing the single locational coordinate moving to various positions on the glenoid 308 corresponding to various calculated center of rotation locations. In another example, mapping one or more center of rotation locations includes calculating a linear distance 312, or delta, between two locations, such first center of rotation location 302 and the second center of rotation location 304 to numerically quantify a migration in the center of rotation of the shoulder joint 104.

As previously set out above, a migration in the center of rotation location of a joint over time can be an indication of a number of issues, such as soft tissue no longer properly constraining the shoulder joint 104, significant erosion of the glenoid fossa of the scapula 111, or other damage to prosthetic implants of a replacement joint. As such, further quantitative analysis can be conducted beyond the calculation and comparison of estimated center of rotation locations. For example, the fit of data points (e.g., locational coordinates) 316, such as representative of a subsequently collected data set, can be compared to the best fit model 300 calculated from data points 314, such as representative of a first data set. A significant deviation from the best fit model 300 can indicate various issues with the shoulder joint 104.

An acceptable or otherwise healthy shoulder joint can generally be indicated by a data set having a standard deviation, or R² value, relative to the best fit model 300 of about 0.85-0.89, 0.9-0.99, or 1.0, such as shown by data points (e.g., locational coordinates) 314. As such, a higher standard deviation can indicate some aspect of shoulder degeneration is occurring, such as indicated by a value of about 0.6-0.69, 0.7-0.79, or 0.8-0.85, such as shown by data points 316. Moreover, determining how the goodness-of-fit starts to deviate or significantly fall away from the best fit model 300 over time can be used to identify regions of joint weakness. Such an approach can allow the sensing system 100 to identify and store a known region of weakness of the shoulder joint 104, such as to provide the patient 106 with cautionary alerts during certain joint movements or excursions.

Additionally, the overall locational spread or distribution of data points included in a data set can be manually studied to identify trends, such as a visible drift or migration in plurality of data points between data sets. This can be helpful in identifying a specific deformity of the shoulder joint 104. For example, if various data points (e.g., locational coordinates) generally deviate from a first data set in a specific or linear direction, an indication of glenoid implant wear or damage, or degradation of a particular soft tissue can be inferred. If various data points generally drift in a generally broader and posterior direction, scapular notching or impingement can be inferred. The above approach can also be used to help identify or confirm a region of weakness or instability of the shoulder joint 104.

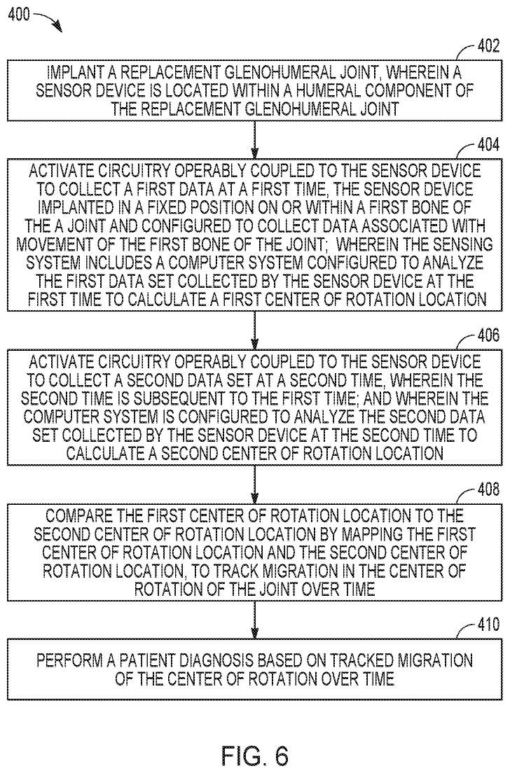

FIG. 6 illustrates a illustrates a flowchart showing a method 400 for tracking a center of rotation of a joint, in accordance with at least one example of the present application. The steps or operations of the method 400 are illustrated in a particular order for convenience and clarity. The discussed operations can be performed in parallel or in a different sequence without materially impacting other operations. The method 400 as discussed includes operations that can be performed by multiple different actors, devices, and/or systems. It is understood that subsets of the operations discussed in the method 400 can be attributable to a single actor device, or system, and could be considered a separate standalone process or method.

The method 400 can include operation 402. Operation 402 includes implanting a replacement glenohumeral joint, wherein a sensor device is located within a humeral component of the replacement glenohumeral joint. For example, in preparation for a total shoulder replacement procedure, one or more sensor devices can be located within, or on, an implant of a patient, such as a humeral component of a prosthetic shoulder joint. The humeral component, including the sensor device, can then be implanted into a patient.

The method 400 can include operation 404. Operation 404 includes activating circuitry operably coupled to the sensor device to collect a first data set at a first time, the sensor device implanted in a fixed position on or within a first bone of the a joint and configured to collect data associated with movement of the first bone of the joint; wherein the sensing system includes a computer system configured to analyze the first data set collected by the sensor device at the first time to calculate a first center of rotation location. Operation 404 can include moving a limb associated with the joint through a range of motion of the joint.

For example, the sensor device can generate data corresponding to motion of the first bone, during a specified timeframe, to collect a first data set. Activation of the sensor device can be via a user input to a user interface of the computer system, or via an input to an intermediary device, in communication with the sensor device. In some examples, the computer system can be a smartphone or a mobile device. The computer system, or other intermediary devices such as a transmitter device, can periodically retrieve data sets from the sensor device. Data from additional sensor devices implanted in a fixed position on or within the first bone, such as a second sensor device, can also be aggregated with data from the sensor device, such that the first data set includes data from more than one source.

In some examples, the first data set can include data from a second sensor device located in a fixed position on or within a second bone of the joint, such as a glenoid. The second sensor device can have a lower sampling rate relative to the sensor device, such as sensor device 102, and can generate data that does not drift over time. In some examples, the second sensor device can be the second sensor device 118. In other examples, the sensor device can be a third sensor device. The second sensor device can be in accordance with the sensor device 102 or can be any variety of other active or passive position-sensing devices. The computer system can thereby reference a geospatial position of the second bone (e.g., glenoid) of the joint in calculating the first center of rotation location. This can improve accuracy by helping to mitigate positional drift by reducing noise or errors in acceleration data generated during movement of the first bone (e.g., humerus or prosthetic humeral component), through a range of motion of the joint. The computer system can utilize various methods and algorithms to calculate the first center of rotation from the first data set.

The method 400 can include operation 406. Operation 406 includes activating circuitry operably coupled to the sensor device to collect a second data set at a second time, wherein the second time is subsequent to the first time; and wherein the computer system is configured to analyze the second data set collected by the sensor device at the second time to calculate a second center of rotation location. Operation 406 can be similar to operation 404 discussed above, except in that the second data set is collected during a specified timeframe subsequent to the first data set. Operation 406 can further include periodically activating circuitry operably coupled to the sensor device to collect an additional data set at a time subsequent to at least the first time, such as to further track migration in the center of rotation of the shoulder joint by calculating additional center of rotation locations.

The method 400 can include operation 408. Operation 408 includes comparing the first center of rotation location to the second center of rotation location by mapping the first center of rotation location and the second center of rotation location, to track migration in the center of rotation of the joint over time. For example, the computer system can compare the first center of rotation location and the second center of rotation by mapping the first center of rotation location and the second center of rotation on a graph or graphical representation of a glenoid. The graphical representation can be displayable to a user on a user interface (e.g., display screen) of the computer system.

The computer system can also calculate a delta, such as a linear distance, between the first center of rotation location and the second center of rotation location, to further illustrate migration in the center of rotation location of a joint over time. Operation 408 can further include comparing additional center of rotation locations to at least one of the first center of rotation location or the second center of rotation location by mapping the additional center of rotation locations, such as to further track migration in the center of rotation of the joint over time.

The operation can optionally include operation 410. Operation 410 includes performing a patient diagnosis based on tracked migration of the center of rotation over time. For example, the computer system can compare any of the first center of rotation location, second center of rotation location, or any additional center of rotation location to track a migration in the center of rotation of a joint over time. A physician can then study the tracked migration, such as to diagnose a deformity and recommend a course of correction action for a patient's shoulder joint.

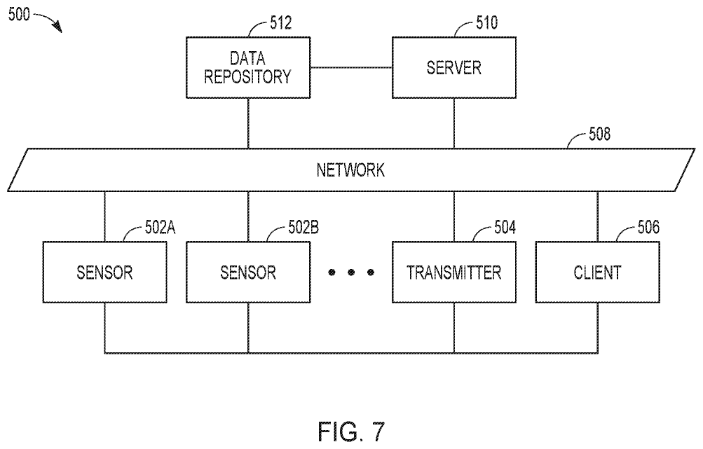

FIG. 7 illustrates an example architecture and componentry for a sensing system 500, in accordance with at least one example of the present application. The sensing system 500 can include any of sensor devices 502A or 502B, a transmitter device 504, a client 506, a network 508, a server 510, and a data repository 512.

The sensor devices 502A and 502B can be any of the sensor devices, such as the sensor device 102 or the second sensor device 118, employed in examples according to this disclosure but can also be or include other suitable sensors. The sensor devices 502A and 502B can be implanted within a patient. The sensor devices 502A and 502B can include a number of different sensors, sensor arrays, including integrated computer-readable storage media or processor(s), as described in detail herein. The transmitter device 504 can be any of the transmitter devices, such as the transmitter device 120 employed in examples according to this disclosure. The transmitter device 504 can be a patient, clinician, or healthcare provider electronic intermediary device for monitoring or otherwise collecting data locally or remotely from the sensor devices 502A and 502B, and transmitting data to, or otherwise communicating with, the server 510 and the data repository 512 via the network 508.

The client 506 data can include an analytics system for processing and analyzing sensor data. The client 506 can run all or portions of, for example, a mobile app for joint assessment, or software for joint assessment, such as for tracking migration in the center of rotation of a joint. The client 506 can be patient, clinician, or healthcare provider electronic devices for monitoring or otherwise collecting data locally or remotely from the sensor devices 502A and 502B, and collecting data from, or otherwise communicating with, the server 510 and the data repository 512 via the network 508. The client 506 can include any number of different portable electronic mobile devices, including, e.g., cellular phones, personal digital assistants (PDA's), laptop computers, portable gaming devices, portable media players, e-book readers, watches, as well as non-portable devices such as desktop computers.

The client 506 can use applications including built-in applications and/or third-party applications. Examples of representative built-in applications can include, but are not limited to, a contacts application, a browser application, a book reader application, a location application, a media application, a messaging application, and/or a game application. Third party applications can include any of the built-in applications as well as a broad assortment of other applications. In a specific example, a third-party application (e.g., an application developed using the Android™ or iOS™ software development kit (SDK) by an entity other than the vendor of the particular platform) can be mobile software running on a mobile operating system such as iOS™, Android™, Windows® Phone, or other mobile operating systems.

The client 506 can include one or more input/output devices configured to allow user interaction with one or more programs. In one example, the client 506 can run a web browser that accesses/executes and presents a web application for use by the user of the client. In another example, the client 506 execute an application outside of a web browser, e.g., an operating system specific application that accesses/executes and presents a native OS application for use by the user of the client 506. The network 508 can include one or more terrestrial and/or satellite networks interconnected to provide a means of communicatively connecting the client 506, the sensor devices 502A and 502B, the data repository 512, and the server 510.

In one example, the network 508 is a private or public local area network (LAN) or Wide Area Network (WANs). The network 508 can include both wired and wireless communications according to one or more standards and/or via one or more transport mediums. In one example, the network 508 includes wireless communications according to one of the 802.11 or Bluetooth specification sets, or another standard or proprietary wireless communication protocol. The sensor devices 502A and 502B, the transmitter device 504, client 506, the server 510, and the data repository 512 and are configured to communicate with one another and to execute functions alone or in conjunction with one another over the network 508.

The network 508 can also include communications over a terrestrial cellular network, including, e.g., a GSM (Global System for Mobile Communications), CDMA (Code Division Multiple Access), EDGE (Enhanced Data for Global Evolution) network. Data transmitted over the network 508, e.g., from the sensor devices 502A and 502B to the client 506 and/or to the data repository 512 and the server 510 can be formatted in accordance with a variety of different communications protocols. For example, all or a portion of the network 508 can be a packet-based, Internet Protocol (IP) network that communicates data in Transmission Control Protocol/Internet Protocol (TCP/IP) packets, over, e.g., Category 5, Ethernet cables.

The server 510 can store and execute data associated with external parties, including, for example, implant manufacturers or healthcare providers. The data repository 512 can be associated with and used for multiple data storage functions. The data repository 512 can be communicatively (e.g., operably, electrically) connected to the transmitter device 504, the client 506, the server 510, and the data repository 512, via network 508. The server 510 can be any of several different types of network and/or computing devices. The examples of the server 510 include a data processing appliance, web server, specialized media server, personal computer operating in a peer-to-peer fashion, or another type of networked device.

Additionally, although example sensing system 500 of FIG. 7 includes one server 510, other examples include a number of collocated or distributed servers configured to process data, surgical plans, etc. individually or in cooperation with one another. Although the server 510 and the data repository 512 are illustrated as separate components in example sensing system 500 of FIG. 7, in other examples, the components can be combined, or each can be distributed amongst more than one device. The server 510 can host and execute portions or all of the surgical planning and assessment system. Additionally, the server 510 or another server or other device connected thereto can include a data analytics system for processing and analyzing sensor data, surgical plans, and other information relevant to surgical planning and post-operative assessment.

The data repository 512 can include, e.g., a standard or proprietary electronic database, or other data storage and retrieval mechanism. In one example, data repository 808 includes one or more databases, such as relational databases, multi-dimensional databases, hierarchical databases, object-oriented databases, or one or more other types of databases. The data repository 512 can be implemented in software, hardware, and combinations of both. In one example, the data repository 512 include proprietary database software stored on one of a variety of computer-readable storage mediums on a data storage server or cloud database connected to the network 508 and configured to store data such as measured or collected sensor data or other information, including aggregated sensor data such as from the sensor devices 502A and 502B.

Storage media included in or employed in cooperation with the data repository 512 can include, e.g., any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. The data repository 512 can be employed to store sensor data. Additionally, the data repository 512 can store and retrieve data or other information from analytics executed on sensor data or a surgical plan, as well as data and other information related to patient population modeling.

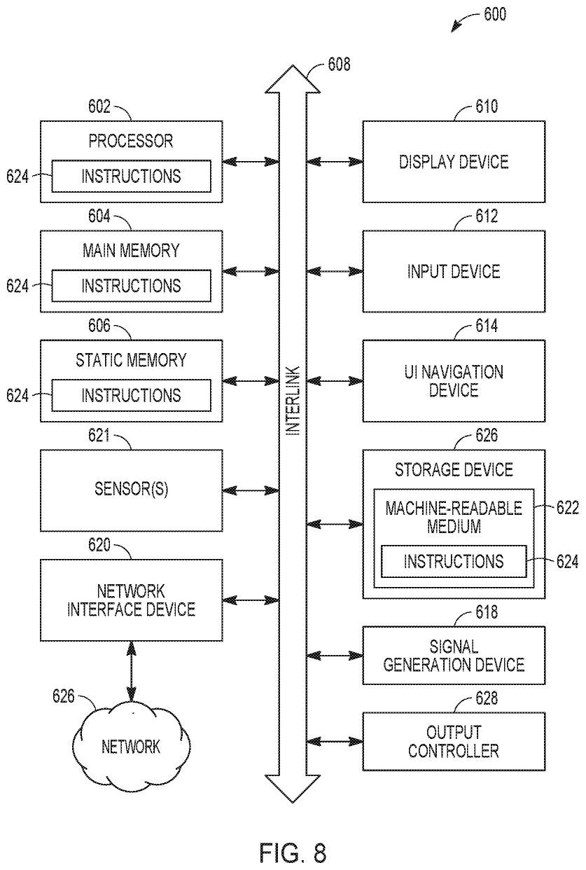

FIG. 8 illustrates a block diagram of an example machine 600 upon which any one or more of the techniques discussed herein can perform in accordance with some embodiments. In alternative embodiments, the machine 600 can operate as a standalone device or can be connected (e.g., networked) to other machines. In a networked deployment, the machine 600 can operate in the capacity of a server machine, a client machine, or both in server-client network environments. In an example, the machine 600 can act as a peer machine in peer-to-peer (P2P) (or other distributed) network environment.

The machine 600 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 can include a hardware processor 602 (e.g., a central processing unit (CPU), a graphics processing unit (GPU), a hardware processor core, or any combination thereof), a main memory 604 and a static memory 606, some or all of which can communicate with each other via an interlink (e.g., bus) 608. The machine 600 can further include a display unit 610, an alphanumeric input device 612 (e.g., a keyboard), and a user interface (UI) navigation device 614 (e.g., a mouse). In an example, the display unit 610, input device 612 and UI navigation device 614 can be a touch screen display. The machine 600 can additionally include a storage device (e.g., drive unit) 616, a signal generation device 618 (e.g., a speaker), a network interface device 620, and one or more sensors 621, such as a global positioning system (GPS) sensor, compass, accelerometer, or other sensors. The machine 600 can include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 can include a machine readable medium 622 on which is stored one or more sets of data structures or instructions 624 (e.g., software) embodying or utilized by any one or more of the techniques or functions described herein. The instructions 624 can also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 can constitute machine readable media.

While the machine readable medium 622 is illustrated as a single medium, the term “machine readable medium” can include a single medium or multiple media (e.g., a centralized or distributed database, or associated caches and servers) configured to store the one or more instructions 624. The term “machine readable medium” can include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples can include solid-state memories, and optical and magnetic media.

The instructions 624 can further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 can include one or more physical jacks (e.g., Ethernet, coaxial, or phone jacks) or one or more antennas to connect to the communications network 626.

In an example, the network interface device 620 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

The foregoing systems and devices, etc. are merely illustrative of the components, interconnections, communications, functions, etc. that can be employed in carrying out examples in accordance with this disclosure. Different types and combinations of sensor or other portable electronics devices, computers including clients and servers, implants, and other systems and devices can be employed in examples according to this disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided.

Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### NOTES AND EXAMPLES

Example 1 is a sensing system for tracking a center of rotation of a joint, comprising: a computer system including: processing circuitry configured to perform operations including: retrieve a first data set collected by a sensor device, the sensor device configured to be implanted into a patient in a fixed location on or within a first bone of the joint, the sensor device configured to collect data associated with movement of the first bone of the joint at a first time; retrieve a second data set collected by the sensor device at a second time, wherein the second time is subsequent to the first time; analyze the first data set and the second data set to calculate a first center of rotation location and a second center of rotation location; and compare the first center of rotation location to the second center of rotation location to track migration in the center of rotation of the joint over time.

In Example 2, the subject matter of Example 1 includes, wherein the joint is a glenohumeral joint, and wherein the sensor device is implanted within a humerus of the patient.

In Example 3, the subject matter of Examples 1-2 includes, wherein the joint is a replacement glenohumeral joint, and wherein the first sensor device is located within a humeral component of the replacement glenohumeral joint extending on or within a humerus.

In Example 4, the subject matter of Examples 1-3 includes, wherein the first data set includes acceleration data and rate of rotation data that corresponds to movement of a limb associated with the joint through a range of motion of the joint.

In Example 5, the subject matter of Examples 1-4 includes, wherein the sensor device is configured to periodically collect an additional data set, the additional data set collected at a time subsequent to the first time.

In Example 6, the subject matter of Example 5 includes, wherein the processing circuitry is configured to periodically retrieve and analyze the additional data set to calculate an additional center of rotation location, and compare the additional center of rotation location to at least one of the first center of rotation location or the second center of rotation location, to track migration in the center of rotation of the joint over time.

In Example 7, the subject matter of Examples 5-6 includes, wherein the computer system is a smartphone or a mobile device including a user interface, the user interface operable to receive a user input to selectively control one or more operations of the processing circuitry, including selectively generating the additional center of rotation location and storing the additional center of rotation location.

In Example 8, the subject matter of Example 7 includes, wherein the processing circuitry is operable to, via a user input, map the first, second, and additional center of rotation locations to display relative locations of the first, second, and additional center of rotation locations to user on the user interface.

Example 9 is a sensing system for tracking a center of rotation of a joint, comprising: a sensor device configured to be implanted into a patient in a first fixed position on or within a first bone of the joint, the sensor device configured to collect first sensor device data associated with movement of the first bone of the joint, the sensor device including: an accelerometer configured to produce acceleration data; a gyroscope configured to produce rate of rotation data, wherein the first sensor data includes, the acceleration data and the rate of rotation data; a second sensor device configured to be implanted into a patient in a second fixed position on or within a second bone of the joint, the second sensor device configured to collect second sensor device data associated with movement of the second bone of the joint; and a computer system including: processing circuitry configured to perform operations including: retrieve a first data set collected at a first time, the first data set including the first sensor device data and the second sensor device data; and retrieve a second data set collected at a second time, the second data set including the first sensor device data and the second sensor device data, wherein the second time is subsequent to the first time; analyze the first data set and the second data set to generate a first center of rotation location and a second center of rotation location; compare the first center of rotation location to the second center of rotation location by mapping the first center of rotation location and the second center of rotation location, to track migration in the center of rotation of the joint over time.

In Example 10, the subject matter of Example 9 includes, a third sensor device configured to be implanted into a patient in a different fixed position on or within a first bone of the joint, relative to the first sensor device.

In Example 11, the subject matter of Examples 9-10 includes, wherein the first sensor device and the second device are configured to periodically collect an additional data set; and wherein the processing circuitry is configured to periodically retrieve and analyze the additional data set to generate an additional center of rotation location, and compare the additional center of rotation location to at least one of the first center of rotation location or the second center of rotation location.

In Example 12, the subject matter of Examples 9-11 includes, wherein mapping the first center of rotation location and the second center of rotation location includes color coding the first center of rotation location differently than the second center of rotation location.

In Example 13, the subject matter of Examples 9-12 includes, wherein mapping the first center of rotation location and the second center of rotation location includes calculating a linear distance between the first center of rotation location and the second center of rotation location.

In Example 14, the subject matter of Examples 9-13 includes, wherein mapping the first center of rotation location and the second center of rotation location includes generating a moving graphical representation illustrating migration of the center of rotation over time, the moving graphical representation displayable to a user on a display device of the computer system.

In Example 15, the subject matter of Examples 9-14 includes, wherein the processing circuitry is configured to analyze the first data set and the second data set to identify a region of weakness or instability of the joint.

In Example 16, the subject matter of Example 15 includes, wherein the computer system is configured to provide an alert to the patient during movements causing the joint to approach or enter the identified region of weakness or instability.

Example 17 is a method for tracking a center of rotation of a joint using a sensing system, the method comprising: activating circuitry operably coupled to a first sensor device to collect a first data set at a first time, the first sensor device implanted in a first fixed position on or within a first bone of a joint and configured to collect data associated with movement of the first bone of the joint; wherein the sensing system includes, a computer system configured to analyze the first data set collected by the first sensor device at a first time to calculate a first center of rotation location; activating circuitry operably coupled to the first sensor device to collect a second data at a second time, wherein the second time is subsequent to the first time; and wherein the computer system is configured to analyze the second data set collected by the first sensor device at the second time to calculate a second center of rotation location; and comparing the first center of rotation location to the second center of rotation location by mapping the first center of rotation location and the second center of rotation location, to track migration in the center of rotation of the joint over time.

In Example 18, the subject matter of Example 17 includes, wherein the method first comprises implanting a replacement glenohumeral joint, wherein the sensor device is located within a humeral component of the replacement glenohumeral joint.

In Example 19, the subject matter of Examples 17-18 includes, wherein activating circuitry operably coupled to the sensor device to collect the first data set and the second data set includes moving a limb associated with the joint through a range of motion of the joint.

In Example 20, the subject matter of Examples 17-19 includes, wherein the sensing system further comprises a second sensor device configured to be implanted in a second fixed position on or within a second bone of the joint, the second sensor device configured to collect data associated with movement of the second bone of the joint, wherein the first data set and the second data set include data from the first sensor device and the second sensor device.

In Example 21, the subject matter of Example 20 includes, activating circuitry operably coupled to the sensor device to periodically collect an additional data set at a time subsequent to the first time; and comparing an additional center of rotation location to at least one of the first center of rotation location and the second center of rotation location, to track migration in a center of rotation of the joint over time.

In Example 22, the subject matter of Examples 20-21 includes, wherein the method further comprises performing a patient diagnosis based on tracked migration of the center of rotation over time.

In Example 23, the subject matter of Examples 20-22 includes, wherein the computer system is a smartphone or a mobile device including a user interface; and wherein activating circuitry operably coupled to the sensor device to periodically collect an additional data set at a time subsequent to the first time is accomplished via at least one user input to the user interface.

Example 24 is at least one machine-readable medium including instructions that, when executed by processing circuitry, cause the processing circuitry to perform operations to implement of any of Examples 1-23.

Example 25 is an apparatus comprising means to implement of any of Examples 1-23.

Example 26 is a system to implement of any of Examples 1-23.

Example 27 is a method to implement of any of Examples 1-23.

What is claimed is:

1. A sensing system for tracking a center of rotation of a joint, comprising:
a computer system including:
processing circuitry configured to perform operations including:
retrieve a first data set collected by a sensor device, the sensor device configured to be implanted into a patient in a fixed location on or within a first bone of the joint, the sensor device configured to collect data associated with movement of the first bone of the joint at a first time;
retrieve a second data set collected by the sensor device at a second time, wherein the second time is subsequent to the first time;
analyze the first data set and the second data set to calculate a first center of rotation location and a second center of rotation location; and
compare the first center of rotation location to the second center of rotation location to track migration in the center of rotation of the joint over time.

2. The system of claim 1, wherein the joint is a glenohumeral joint, and wherein the sensor device is implanted within a humerus of the patient.

3. The system of claim 1, wherein the joint is a replacement glenohumeral joint, and wherein the first sensor device is located within a humeral component of the replacement glenohumeral joint extending on or within a humerus.

4. The system of claim 1, wherein the first data set includes acceleration data and rate of rotation data that corresponds to movement of a limb associated with the joint through a range of motion of the joint.

5. The system of claim 1, wherein the sensor device is configured to periodically collect an additional data set, the additional data set collected at a time subsequent to the first time.

6. The system of claim 5, wherein the processing circuitry is configured to periodically retrieve and analyze the additional data set to calculate an additional center of rotation location, and compare the additional center of rotation location to at least one of the first center of rotation location or the second center of rotation location, to track migration in the center of rotation of the joint over time.

7. The system of claim 5, wherein the computer system is a smartphone or a mobile device including a user interface, the user interface operable to receive a user input to selectively control one or more operations of the processing circuitry, including selectively generating the additional center of rotation location and storing the additional center of rotation location.

8. The system of claim 7, wherein the processing circuitry is operable to, via a user input, map the first, second, and additional center of rotation locations to display relative locations of the first, second, and additional center of rotation locations to user on the user interface.

9. A sensing system for tracking a center of rotation of a joint, comprising:
a sensor device configured to be implanted into a patient in a first fixed position on or within a first bone of the joint, the sensor device configured to collect first sensor device data associated with movement of the first bone of the joint, the sensor device including:
an accelerometer configured to produce acceleration data;
a gyroscope configured to produce rate of rotation data, wherein the first sensor data includes the acceleration data and the rate of rotation data;
a second sensor device configured to be implanted into a patient in a second fixed position on or within a second bone of the joint, the second sensor device configured to collect second sensor device data associated with movement of the second bone of the joint; and
a computer system including:
processing circuitry configured to perform operations including:
retrieve a first data set collected at a first time, the first data set including the first sensor device data and the second sensor device data; and
retrieve a second data set collected at a second time, the second data set including the first sensor device data and the second sensor device data, wherein the second time is subsequent to the first time;
analyze the first data set and the second data set to generate a first center of rotation location and a second center of rotation location;
compare the first center of rotation location to the second center of rotation location by mapping the first center of rotation location and the second center of rotation location, to track migration in the center of rotation of the joint over time.

10. The system of claim 9, further comprising a third sensor device configured to be implanted into a patient in a different fixed position on or within a first bone of the joint, relative to the first sensor device.

11. The system of claim 9, wherein the first sensor device and the second device are configured to periodically collect an additional data set; and wherein the processing circuitry is configured to periodically retrieve and analyze the additional data set to generate an additional center of rotation location, and compare the additional center of rotation location to at least one of the first center of rotation location or the second center of rotation location.

12. The system of claim 9, wherein mapping the first center of rotation location and the second center of rotation location includes color coding the first center of rotation location differently than the second center of rotation location.

13. The system of claim 9, wherein mapping the first center of rotation location and the second center of rotation location includes calculating a linear distance between the first center of rotation location and the second center of rotation location.

14. The system of claim 9, wherein mapping the first center of rotation location and the second center of rotation location includes generating a moving graphical representation illustrating migration of the center of rotation over time, the moving graphical representation displayable to a user on a display device of the computer system.

15. The system of claim 9, wherein the processing circuitry is configured to analyze the first data set and the second data set to identify a region of weakness or instability of the joint.

16. The system of claim 15, wherein the computer system is configured to provide an alert to the patient during movements causing the joint to approach or enter the identified region of weakness or instability.

17. A method for tracking a center of rotation of a joint using a sensing system, the method comprising:
activating circuitry operably coupled to a first sensor device to collect a first data set at a first time, the first sensor device implanted in a first fixed position on or within a first bone of a joint and configured to collect data associated with movement of the first bone of the joint; wherein the sensing system includes a computer system configured to analyze the first data set collected by the first sensor device at a first time to calculate a first center of rotation location;
activating circuitry operably coupled to the first sensor device to collect a second data at a second time, wherein the second time is subsequent to the first time; and wherein the computer system is configured to analyze the second data set collected by the first sensor device at the second time to calculate a second center of rotation location; and
comparing the first center of rotation location to the second center of rotation location by mapping the first center of rotation location and the second center of rotation location, to track migration in the center of rotation of the joint over time.

18. The method of claim 17, wherein the method first comprises implanting a replacement glenohumeral joint, wherein the sensor device is located within a humeral component of the replacement glenohumeral joint.

19. The method of claim 17, wherein activating circuitry operably coupled to the sensor device to collect the first data set and the second data set includes moving a limb associated with the joint through a range of motion of the joint.

20. The method of claim 17, wherein the sensing system further comprises a second sensor device configured to be implanted in a second fixed position on or within a second bone of the joint, the second sensor device configured to collect data associated with movement of the second bone of the joint, wherein the first data set and the second data set include data from the first sensor device and the second sensor device.

21. The method of claim 20, further comprising activating circuitry operably coupled to the sensor device to periodically collect an additional data set at a time subsequent to the first time; and comparing an additional center of rotation location to at least one of the first center of rotation location and the second center of rotation location, to track migration in a center of rotation of the joint over time.

22. The method of claim 20, wherein the method further comprises performing a patient diagnosis based on tracked migration of the center of rotation over time.

23. The method of claim 20, wherein the computer system is a smartphone or a mobile device including a user interface; and wherein activating circuitry operably coupled to the sensor device to periodically collect an additional data set at a time subsequent to the first time is accomplished via at least one user input to the user interface.
